(19) United States
(12) Patent Application Publication
(10) Pub. No.: US 2021/0361375 A1
(43) Pub. Date: Nov. 25, 2021

(54) DISPOSABLE HOOD TO PREVENT SPREAD OF AEROSOLIZED PATIENT DISCHARGE

(71) Applicant: RHINO RHI, LLC, Allentown, PA (US)

(72) Inventors: Michael Hartman, Jim Thorpe, PA (US); Carl Andreas, II, Allentown, PA (US); Michael F. Fella, Fleetwood, PA (US)

(73) Assignee: RHINORHI, LLC, Allentown, PA (US)

(21) Appl. No.: 17/327,681

(22) Filed: May 22, 2021

Related U.S. Application Data
(60) Provisional application No. 63/029,505, filed on May 24, 2020.

(51) Int. Cl. A61B 90/00

(52) U.S. Cl. CPC A61B 90/05

(57) ABSTRACT

A portable protective hood for use during medical procedures to avoid the spreading of aerosolized patient discharges. The protective hood includes an easy to assembly frame having a base plate formed with mounting sockets that can receive the remote ends of plastic ribs that will arch over the base plate. A transparent plastic bag has an elastomeric opening that enables the plastic bag to be placed over the upright ribs and extend down to the base plate. The base plate is shaped to incorporate curved sections that allow the movement of air from the atmosphere into the plastic bag, which is extracted through a connector on the plastic bag for attachment to a vacuum extraction device. The base plate and ribs are formed from a non-porous plastic material that permits cleaning between uses. The base plate and ribs can be placed into a storage configuration when not in operative use.

### CROSS-REFERENCE TO RELATED APPLICATIONS

This application claims domestic priority on U.S. Provisional Patent Application Ser. No. 63/029,505, filed on May 24, 2020, the content of which is incorporated herein by reference.

### FIELD OF THE INVENTION

This invention relates generally to a protective hood that encapsulates the head of a patient to prevent the spread of aerosolized patient discharges during medical procedures and, more particularly, to a disposable hood structure that is easily utilized by both hospital and prehospital personnel to minimize exposure to infectious discharges from patients.

### BACKGROUND OF THE INVENTION

Protective devices have become a vital necessity with the continuing coronavirus pandemic. Medical personnel are exposed to infectious patients, whether that patient is infected with the coronavirus or other diseases, many of which can be fatal. One way the infection is spread is through aerosolized discharges from the patient, such as by sneezing or coughing. An uncontained aerosolized discharge will require a thorough cleaning of the facility, such as a surgical suite or an ambulance, in which the patient is located at the time the aerosolize droplets are discharged. Aerosolized discharges from patients are common with intubation and extubation of an advanced airway supporting device for surgical and other medical procedures.

Over the years, protective hoods have been developed to contain the aerosolized discharges of a patient, but none are economically and effectively disposable and easily implemented or stored, and thus, have not become an effective protective measure that can be commonly deployed. One such device is disclosed in U.S. Pat. No. 4,407,280, granted on Oct. 4, 1983, to W. Edgar Trammell, et al, in which the hood is a molded transparent, bubble-shaped chamber that isolates the head of the patient and includes openings for connection with hoses to permit the input of oxygen or other gases for consumption by the patient. This rigid, molded bubble-shaped chamber is cumbersome to deploy and to store and is not conducive to disposal as a single use apparatus.

A similar apparatus to the Trammell transparent, bubble-shaped chamber is found in U.S. Pat. No. 4,444,183, granted to David Heckendorn on Apr. 24, 1984, in which the enclosure for the patient's head is a box-like structure with a gas input port and an exhaust port incorporated into the planar walls of the enclosure apparatus. Neither the Trammell nor Heckendorn devices facilitate access to the patient's head for procedures such as intubation and extubation of the endotracheal tube.

Similar hood devices have also been developed for application of and scavenging medicinal agents administered to the patient as an aerosol spray. These devices also employ rigid transparent hoods that fit over and around the patient's head to control the application of the medicinal agent to the patient. Such devices can be found, for example, in U.S. Pat. No. 4,949,714, granted to Robert Orr on Aug. 21, 1990, in which the enclosure device is provided with a gas input port and an exhaust port to control the application of medicinal agents, particularly anesthesia. A similar medicinal scavenging devices is found in U.S. Pat. No. 6,076,524, issued on Jun. 20, 2000, to Stephen Corn in which the enclosure devices is formed from a gas impermeable transparent plastic film, such as polyvinyl chloride (PVC), that creates a bag the closely encapsulates the patient's head. A gas input port and exhaust port closely control the volume of the bag, but an emergency tab and score lines are provide to permit a quick release of the bag from the patient in the event of an emergency.

In U.S. Pat. No. 5,819,728, issued on Oct. 13, 1998, to Scott Ritchie, a gas treatment hood is disclosed in which the hood is shaped similar to that of a diving bell that fits over the head of the patient and is captured with a neck ring that seals the hood with respect to the patient. Gas inlet and gas discharge tubes control the interior environment of the patient's hood, which is formed from a soft, flexible polyvinyl sheet. A similar head enclosing treatment hood is disclosed in U.S. Pat. No. 6,854,459, granted to Gerald Cox on Feb. 15, 2005, for the treatment of respiratory ailments. An alternative embodiment of the Cox treatment hood includes a frame of a ring and vertical ribs that support the flexible film hood and prevent the hood from collapsing onto the patient's head.

A collapsible, transportable isolation chamber is disclosed in U.S. Pat. No. 6,461,290, granted on Oct. 8, 2002, to David Reichman, et al, in which an elongated collapsible structure formed of a thin PVC film housing bonded to circular plastic ribs that will expand from a collapsed state to an elongated state for enclosure around and isolation of a patient. The housing is provided with multiple glove ports for accessing the patient once sealed within the isolation chamber. Air inlet and outlet ports filter the air through the chamber. A similar isolation chamber is disclosed in U.S. Pat. No. 7,757,689, issued on Jul. 20, 2010, to Wang-Hsing Chang, in which the chamber is formed to be inflatable from a storage orientation to create the isolation chamber.

None of these known prior art references disclose a hood device that can be stored in a compact configuration and deployed quickly and easily to protect medical personnel working with a patient from being exposed to any discharge from the patient containing aerosolized pathogens. Furthermore, none of these known prior art devices contain the capability of permitting access to the patient's head after being encapsulated in order to accomplish certain medical procedures, including intubation and extubation of the endotracheal tube from the patient's throat.

Accordingly, it would be desirable to provide a protective hood that can be easily deployed by first responders and other medical personnel to encapsulate the head of the patient and any aerosolized discharges from that patient that may contain harmful pathogens. It would also be desirable to provide a protective hood that will enable convenient access to the patient for procedures such as intubation and extubation of an endotracheal tube for the patient.

### SUMMARY OF THE INVENTION

It is an object of this invention to overcome the disadvantages of the prior art by providing a disposable protective hood to prevent the spread of aerosolized patient discharges during medical procedures.

It is another object of this invention to provide a protective hood that can be used with a patient to prevent the spread of aerosolized patient discharges during medical procedures.

It is a feature of this invention that the patient is enclosed within a transparent plastic bag connected to a vacuum discharge apparatus to remove air and aerosolized patient discharges during medical procedures.

It is an advantage of this invention that the protective hood allows a flow of air from along the base plate to keep the plastic bag from collapsing.

It is another feature of this invention that the frame of the protective hood includes a base plate and removable ribs mounted on the base plate and arching over the surface of the base plate to encompass a patient when a transparent plastic bag is positioned over the assembled frame.

It is another advantage of this invention that the transparent plastic bag includes an elastomeric opening that will allow the transparent plastic bag to stretch over the assembled ribs to the base plate.

It is still another advantage of this invention that the elastomeric opening of the transparent plastic bag enables a care giver to access the patient being treated without requiring the removal of the protective hood.

It is still another feature of this invention that the base plate can be provided with a foam pad to add to the comfort of the patient.

It is yet another advantage of this invention that the base plate and ribs can be formed from a non-porous plastic material, such as polyvinyl chloride (PVC), so that the base plate and ribs can be cleaned and reused for subsequent patient treatments.

It is yet another feature of this invention that the base plate and ribs can be mounted together into a collapse storage configuration when not in use.

It is still another advantage of this invention that the transparent plastic bag can be disposed after being used for patient treatment.

It is still another object of this invention to provide a protective hood that will prevent the spread of aerosolized patient discharges during medical procedures, which is easy to use, simple to manufacture and assembly, and effective to use.

These and other objects, features and advantages will be found by providing a portable protective hood that can be used for medical procedures and avoid the spreading of aerosolized patient discharges. The protective hood includes an easy to assembly frame having a base plate formed with mounting sockets that can receive the remote ends of plastic ribs that will arch over the base plate. A transparent plastic bag has an elastomeric opening that enables the plastic bag to be placed over the upright ribs and extend down to the base plate. The base plate is shaped to incorporate curved sections that allow the movement of air from the atmosphere into the plastic bag, which is extracted through a connector on the plastic bag for attachment to a vacuum extraction device. The base plate and ribs are formed from a non-porous plastic material that permits cleaning between uses. The base plate and ribs can be placed into a storage configuration when not in operative use.

### BRIEF DESCRIPTION OF THE DRAWINGS

The advantages of this invention will become apparent upon consideration of the following detailed disclosure of the invention, especially when taken in conjunction with the accompanying drawings wherein:

FIG. 1 is a schematic perspective view of an assembled frame for a protective hood incorporating the principles of the instant invention;

FIG. 2 is a schematic representation of a collapsible transparent plastic bag forming the exterior shell of the protective hood when applied to the assembled frame as depicted in FIG. 1;

FIG. 3 is a schematic perspective view of the protective hood fully assembled and placed around a representative patient, shown in dashed lines;

FIG. 4 is a schematic plan view of the base plate for the frame of the protective hood;

FIG. 5 is a schematic plan view of the mounting bracket for supporting the unassembled frame from a support structure, such as a wall of a surgical suite or other vertical surfaces;

FIG. 6 is a schematic elevational view of the mounting bracket shown in FIG. 5;

FIG. 7 is a schematic vertical elevational view of the unassembled frame of the protective hood in a collapsed configuration and mounted on a vertical support by the mounting bracket;

FIG. 8 is a schematic perspective view of the protective hood fully assembled and placed around a representative patient, shown in dashed lines, similar to that of FIG. 3, but depicting the flexibility of the elastomeric banded opening of the transparent plastic bag placed over the assembled frame in accessing the patient's head for medical procedures;

FIG. 9 is a schematic plan view of an alternative configuration for the base plate incorporating the principles of the instant invention;

FIG. 10 is a schematic perspective view of a polyurethane foam pad that is secured to the base plate shown in FIG. 9;

FIG. 11 is a schematic elevational view of an assembled frame for the protective hood utilizing the alternative base plate and polyurethane pad depicted in FIGS. 9 and 10; and

FIG. 12 is a schematic perspective view of the unassembled frame of the protective hood with the ribs positioned in a storage position on the base plate.

### DETAILED DESCRIPTION OF THE PREFERRED EMBODIMENT

Referring to the drawings, a protective hood encapsulating the head of a patient to protect medical personnel from aerosolized harmful pathogens expelled by the patient can best be seen. Current commercially available products that protect medical personnel from aerosolized discharges from the patient are rigid, heavy, and are not disposable or provide for single patient usage. Other such products do not have capabilities for the removal of aerosolized harmful pathogens by attaching a suction device to the enclosure apparatus. Rather than provide a cumbersome, inconveniently deployed apparatus, the protective hood incorporating the principles of the instant invention can be converted to a collapsed, stored configuration into an operative, deployed configuration in a matter of a few seconds.

The protective hood 10 includes a plastic frame 12 that is formed with a base plate 13 formed in a dog-bone shape for purposes to be defined in greater detail below, and a pair of tubular, curved ribs 15 that are detachably engaged with mounting sockets 14 formed on the base plate 12 at the four rounded corners thereof. The mounting sockets 14 can be sonic welded to the base plate 13 or integrally formed therewith, such as by injection molding techniques. The curved, tubular ribs 15 have a round diameter configuration, which enables convenient cleaning of the ribs 15, as will be described in greater detail below, and are pre-formed in a semi-circular overall shape that is slightly shape retentive to permit convenient storage, as is further described below. The base plate 13 and ribs 15 are preferably constructed from a non-porous plastic material, such is polyethylene or polyvinyl chloride (PVC) to permit a thorough cleaning thereof, which allows the base plate 13 and ribs 15 to be reused from one patient to another.

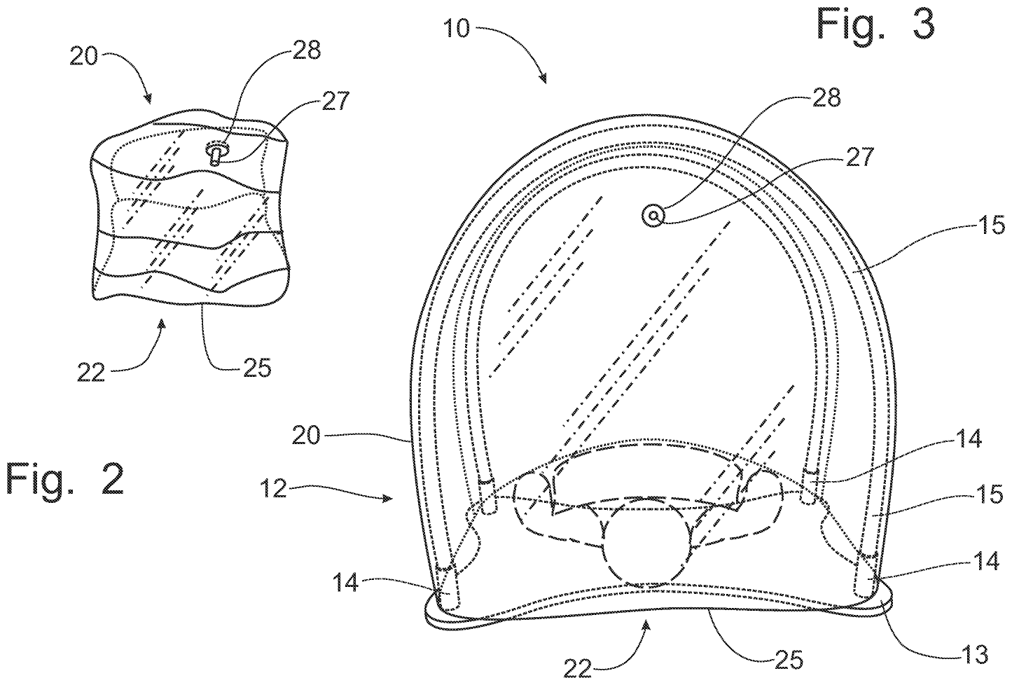

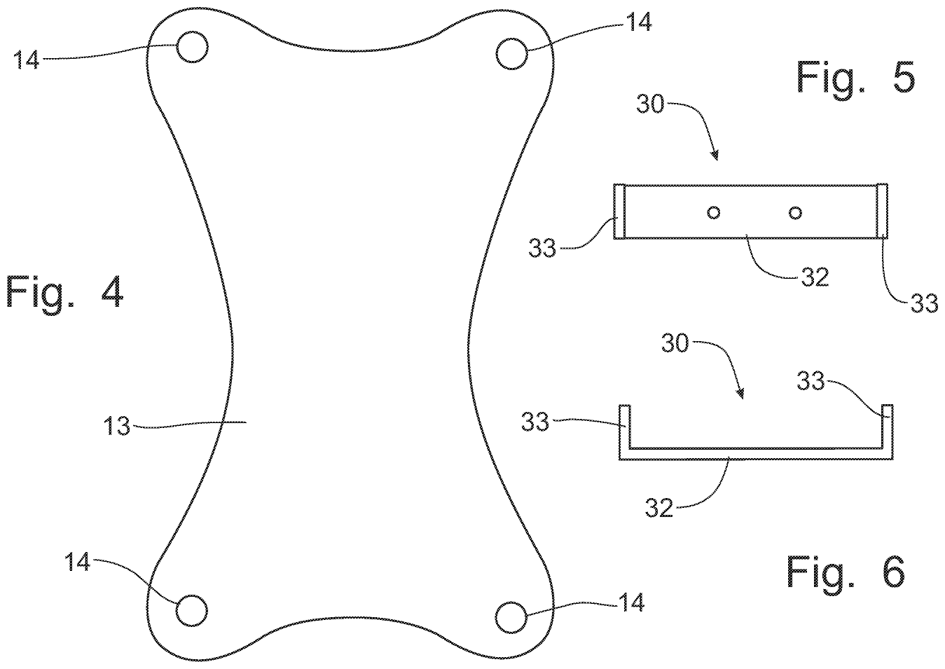

The hood portion of the protective hood 10 is formed by a transparent plastic bag 20 that has an opening 22 formed with a non-latex elastomeric band 25 around the circumference that permits expansion of the diameter of the opening from a collapsed configuration, as represented in FIG. 2, to the size of the opening 22 needed to be placed around the ribs 15. The elastomeric band 25 is positioned adjacent the base plate 13 and spread about the mourning sockets 14, as is depicted in FIG. 3. One skilled in the art will recognize that the generally straight line between any two adjacent mounting sockets 14 passes over a curved section of the dog-bone shaped base plate 13, which allows the passage of air underneath the elastomeric band 25 and into the plastic bag 20. Thus, all four sides of the base plate 13 are formed with inwardly curved sections that allow the passage of air under the elastomeric band 25.

The transparent plastic bag 25 is also equipped with an exhaust port 27 connected to a one-way valve 28 that will permit air to be evacuated from the interior of the transparent plastic bag 20, but only with the application of negative pressure to the exhaust port 27. Thus, the harmful pathogens in any aerosolized discharge from the patient within the protective hood 10 can be exhausted to a filter (not shown) when a vacuum source is attached to the discharge port, but the contaminated air within the protective hood 10 cannot escape through the discharge port 27 because of the valve 28. Preferably, the exhaust port 27 is a standard barbed configuration known in the medical arts. The replacement of the air within the protective hood 10 drawn out by the vacuum source (not shown) attached to the discharge port 27 is accomplished by the aforementioned gaps between the elastomeric band 25 and the inwardly curved portions of the base plate 13, which enables air to flow along the base plate 13 into the protective hood 10. Alternatively, the transparent plastic bag 20 can also be provided with a second port (not shown) that can be used to inject oxygen or other needed gases for the benefit of the patient.

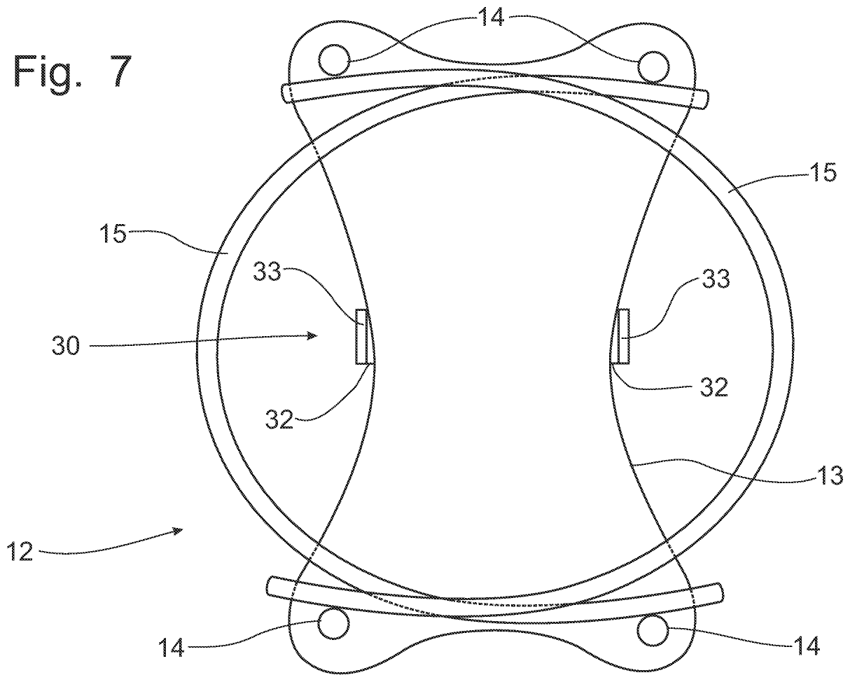

As noted above, the ribs 15 are formed as circular diameter tubular members that are easy to disinfect and clean for re-use with a subsequent patient. The transparent plastic bag 20 is disposable and when use is finished, the plastic bag 20 will be properly disposed, then replaced by a new, preferably sterile, transparent plastic bag 20. Meanwhile, the frame 12 of the protective hood 10 can be stored as depicted in FIG. 7. With the ribs 15 disengaged from the mounting sockets 14, the shape retentive semi-circular hoops formed by the ribs 15 will fit between longitudinally opposite mounting sockets 14 if depressed slightly against the inherent spring action of the curved rib 15. Then, when the ribs 15 try to return to the pre-formed shape, the ribs 15 press against the longitudinally opposed mounting sockets 14 and remain as positioned until the ribs 15 are pulled away from the base plate 13 to be re-assembled.

Preferably, the disassembled, stored configuration of the frame 12 can be supported on a vertical surface by a support bracket 30 formed with a longitudinal base member 32, and a pair of opposing perpendicular legs 33. The dimension of the spacing between the opposing perpendicular legs 33 is slightly greater than the minimum spacing between the opposing longitudinal curved sections of the base plate 13. With the support bracket 30 attached to a wall, or other vertical support surface, such as by a pair of screws (not shown), the base plate 13 with the two ribs 15 supported between respective longitudinally spaced mounting sockets 14, as is depicted in FIG. 7, can be positioned on the support bracket 30 and allowed to slide down slightly until the increasing dimension of the spacing between the inwardly curved sections equals the dimension of the spacing between the perpendicular legs 33. The stored configuration of the frame 12 will then hang from the support bracket 30 without additional support required. The transparent plastic bag 20 can be supported on the frame 12 as a compressed sterilized package or placed into an adjacent drawer or container with other such packages.

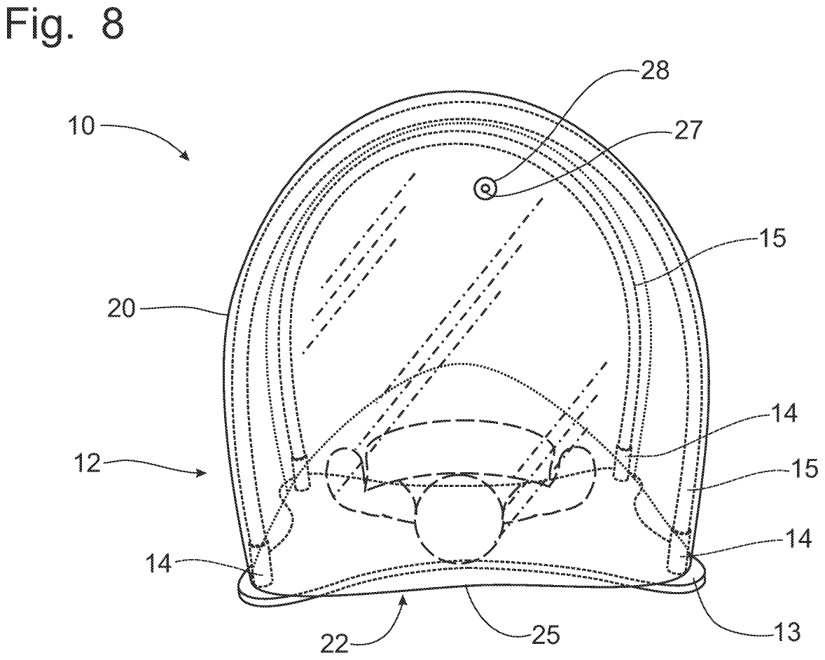

As is best seen in FIG. 3, the elastomeric band 25 around the opening 22 of the transparent plastic bag 20 will deflect over the trunk of the body of the patient, which also leaves a slight gap between the elastomeric band 25, the base plate 13 and the patient to allow the passage of replacement air into the protective hood when a vacuum source is connected to the discharge port 27. This same elastomeric band 25 also has sufficient stretch capabilities to allow medical personnel to access the patient's head over top of the truck of the patient by lifting the elastomeric band 25, as is represented in FIG. 8. Thus, the operative configuration of the protective hood 10 with the elastomeric band around the base plate 13 and over the patient will permit medical personnel to perform an intubation or an extubation of an endotracheal tube from the patient's throat without having to remove the protective hood 10 from the patient. Accordingly, if an aerosolized discharge is encountered from the patient during such medical procedures, the aerosolized harmful pathogens are expressed upwardly and are evacuated through the discharge port 27 and the vacuum source applied thereto.

As a matter of example, the preferred embodiment of the instant invention would dimension the base plate 13 in order to fit within a 24 inch by 24 inch box for shipping. Accordingly, the base plate 13 would have a maximum longitudinal length of about 23.5 inches, and preferably a corresponding maximum width of perhaps about 14.5 inches. The ribs 15 would preferably have a diameter of 0.5 inches to be insertable into the mounting sockets 14 having a ⅝ inch diameter. Each rib 15 would be approximately 50-54 inches long along the curved length of the rib 15 to establish an overall height of the assembled protective hood 10 of about 18 inches.

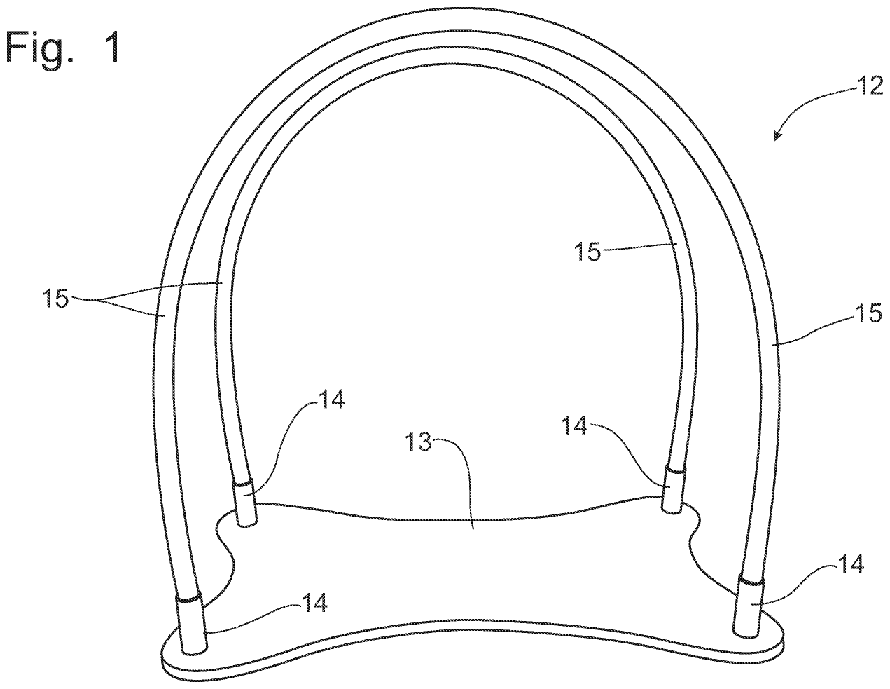

In operation, starting with a stored configuration of the frame 12, as is depicted in FIG. 7 with a compressed, collapsed sterile transparent plastic bag 20, as described above, the frame 12 is first assembled by pulling the stored frame 12 off of the support bracket 30 and removing the ribs 15 from the engagement with the mounting sockets 14. The respective ends of each rib 15 are inserted into a corresponding, longitudinally opposed mounting sockets 14 of the base plate 13, as is depicted in FIG. 1. The base plate 13 is then slide under the upper back portion of the patient and positioned such that the head of the patient is over the base plate 13.

The transparent plastic bag 20 is then removed from the protective packaging, opened up and then pulled over the upright ribs 15 until the elastomeric band 25 is stretched about the mounting sockets 14 on the base plate 13 and across the chest of the patient, as is reflected in FIG. 3. A vacuum source (not shown) is attached to the discharge port 27 to exhaust air from within the protective hood 10. The exhausted air is replaced along the base plate 13 beneath the elastomeric band 25, as is described above. In the event medical personnel need to access the head of the patient, such as for the intubation or extubation of the endotracheal tube from the patient's throat, the elastomeric band 25, such as above the patient's chest, can be further stretched to permit the hands of the person accessing the patient to reach the patient's head.

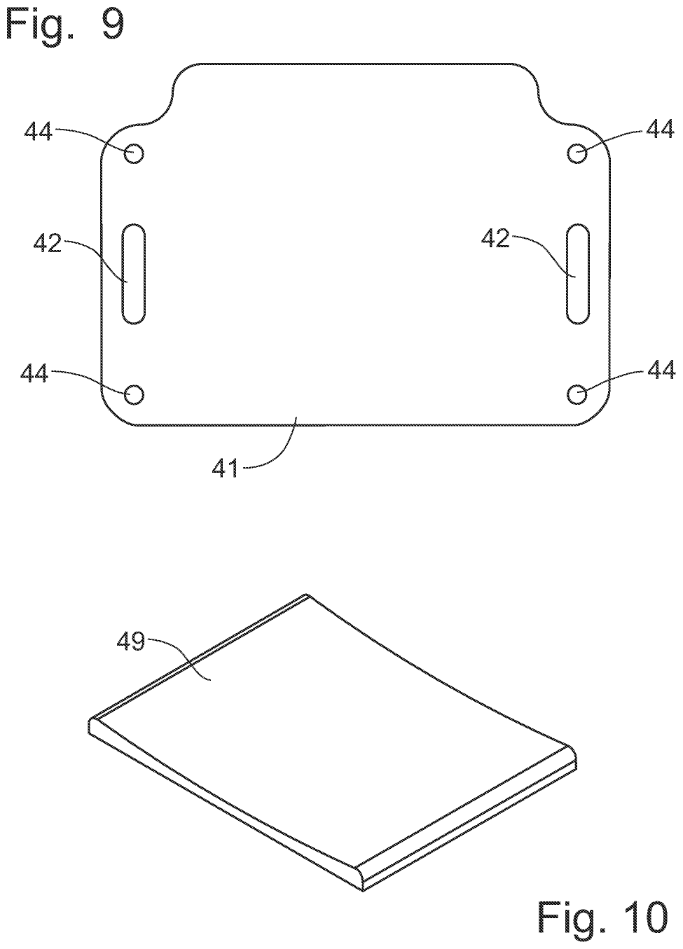

Referring now to FIGS. 9-12, an alternative configuration of the protective hood 10 can best be seen. The frame 40 includes a base plate 41 formed into generally a rectangular shape with handles 42 incorporated into the ends of the base plate 41 to facilitate handling of the protective hood, whether assembled or unassembled. A foam pad 49, preferably formed from polyurethane foam, is secured to the base plate 41 for the comfort of the patent. The foam pad is formed with a vertically curved shape for the comfort of the patient being treated, but also to prevent the transparent bag 20 from sealing against the base plate 41, thus allowing a flow of air from the atmosphere into the transparent bag 20 as is noted above.

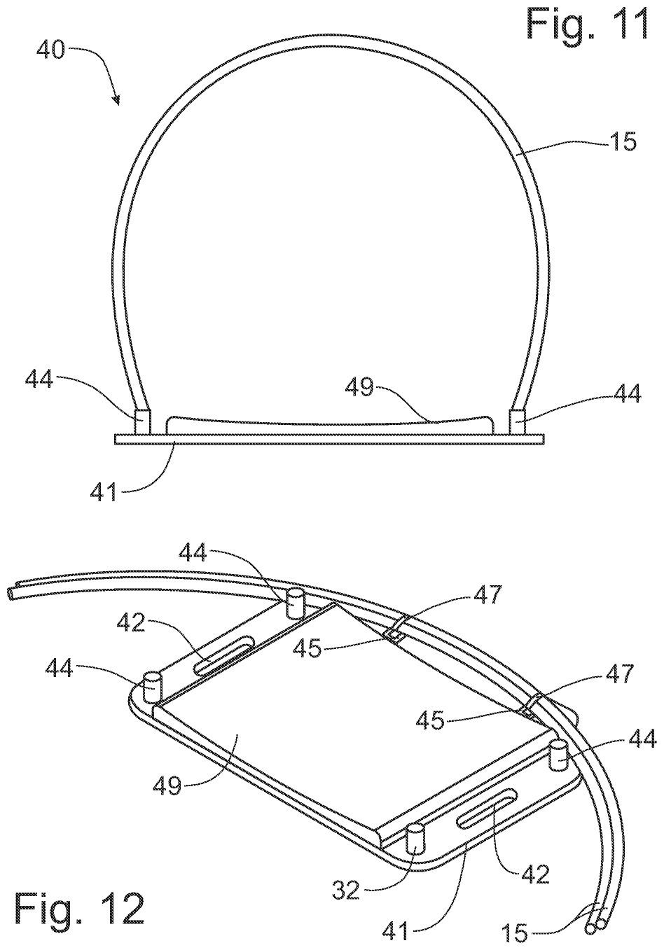

The base plate 41 is also formed with mounting sockets 44 on opposing sides of each of the handles 42 and sized to receive the ends of the ribs 15 in a manner that they extend from one end of the base plate 41 to the other end of the base plate 41, as is depicted in FIG. 11. As a matter of materials, the base plate 41 is preferably formed from a non-porous plastic material, such is polyethylene or polyvinyl chloride (PVC) to permit a thorough cleaning thereof, which allows the base plate 41 and ribs 15 to be reused from one patient to another.

Preferably, the scalloped side of the base plate 41 is formed with a pair of spaced nylon mounts 45 to which are mounted respective connective straps 47, such as hook and loop fasteners that can encircle and restrain the disassembled ribs 15 to create a storage configuration for this alternative embodiment. As is depicted in FIG. 12 the disassembled frame 40 can be collapsed into a storage configuration with the disassembled ribs 15 secured to the base plate 41 by the connective straps 47 and placed into a convenient location for deployment when needed. The transparent plastic bag 20 is replaced with each use, and can be hung from the base plate 41 or the secured ribs 15 for easy access; however, the base plate 41 and ribs 15 are constructed to enable cleaning and redeployment with a new transparent plastic bag 20 for the subsequent use of the protective hood 10.

One skilled in the art will recognize that this protective hood could be first utilized by EMTs when first engaging and transporting a patient with an unknown condition, which potentially could involve harmful pathogens, particularly the novel coronavirus. Thus, the above assembly instructions can be accomplished at the first occurrence of the new patient, or in the ambulance. Due to the nature of the protective hood incorporating the principles of the instant invention, this protective hood 10 installed by EMTs could be left on the patient through the emergency room, or even to surgery, simply by switching appropriate vacuum sources connectable to the exhaust port 27. The protective hood 10 can also be applied in conjunction with general anesthesia laryngeal mask airway cases to remove or reduce aerosolized particles. The protective hood 10 can further be utilized with any pre-hospital or hospital-based aerosolization procedure involving airway management. The transparent plastic bag 20 with evacuation port 27 can be used with vaporization procedures that produce aerosolized particles.

It will be understood that changes in the details, materials, steps and arrangements of parts which have been described and illustrated to explain the nature of the invention will occur to and may be made by those skilled in the art upon a reading of this disclosure within the principles and scope of the invention. The foregoing description illustrates the preferred embodiment of the invention; however, concepts, as based upon the description, may be employed in other embodiments without departing from the scope of the invention.

Having thus described the invention, what is claimed is:

1. An encapsulating protective hood, comprising:
a frame assembly including a base plate and at least one curved rib detachably connected to said base plate and extending in an arc over top of the base plate; and
a transparent collapsible bag having an opening encircled with an elastomeric band permitting the opening to be stretched to cover the frame assembly with the opening placed along said base plate and encapsulating said frame assembly.

2. The protective hood of claim 1 wherein said base member is formed with an inwardly curved section along each side of said base plate.

3. The protective hood of claim 1 wherein said base plate if formed with two mounting sockets for each curved rib, each said mounting socket projecting upwardly from said base plate and being adapted to receive respective ends of said ribs to detachably mount said ribs to said base plate.

4. The protective hood of claim 3 wherein said base plate has four sides, each of which is formed with said inwardly curved section, said mounting sockets being positioned proximate to four corners of said base plate.

5. The protective hood of claim 4 wherein said elastomeric band of said transparent collapsible bag encircles the mounting sockets, overlapping the respective inwardly curved sections to permit a passage of air underneath said elastomeric band and into said transparent bag.

6. The protective hood of claim 5 wherein said transparent collapsible bag include an exhaust port located at a position spaced from said elastomeric band, said exhaust port being adapted for connection to a vacuum source to exhaust air from within said protective hood.

7. The protective hood of claim 6 wherein said ribs are curved tubular members having a circular diameter to facilitate cleaning.

8. A protective hood to prevent a spread of an aerosolized discharge from a patient, comprising:
a frame assembly including a base plate and a pair of curved ribs detachably connected to said base plate and extending in an arc over top of the base plate, said base plate including mounting sockets extending upwardly from said base plate to receive respective ends of each curved rib, said frame assembly being sized to receive an upper portion of the patient, including the patient's head;
a transparent collapsible bag having an opening encircled with an elastomeric band permitting the opening to be stretched to cover the frame assembly with the opening placed along said base plate and encapsulating said frame assembly, said elastomeric band passing over the portion of said patient within said frame assembly; and
an evacuation port secured to said transparent bag for connection to a remote vacuum source to evacuate air and aerosolized discharge from within said transparent collapsible bag.

9. The protective hood of claim 8 wherein said base member is formed with an inwardly curved section along each side of said base plate so that said elastomeric band extends around said mounting sockets and overlaps said inwardly curved sections of said base plate to prevent an airtight seal by said elastomeric band against said base plate.

10. The protective hood of claim 8 wherein said ribs are curved tubular members having a circular diameter to facilitate cleaning.

11. The protective hood of claim 8 wherein said elastomeric band has sufficient stretch to permit a medical attendant to lift the elastomeric band above the patient's chest to gain access to the patient's head.

12. The protective hood of claim 8 wherein said base plate is formed in a generally rectangular shape with a scalloped side that so that said elastomeric band extends around said mounting sockets and overlaps said scalloped side of said base plate to prevent an airtight seal by said elastomeric band against said base plate.

13. The protective hood of claim 12 wherein said base plate has a foam pad secured thereto for comfort of the patient.

14. A method of preventing a spread of an aerosolized discharge from a patient, comprising the steps of:
assembling a protective hood to encapsulate the patient's head, said assembling step including the steps of:
removing a frame assembly from a stored configuration, said frame assembly having a base plate and a pair of curved ribs;
inserting respective ends of said curved ribs into corresponding mounting sockets on said base plate to position said curved ribs in an arched orientation over said base plate;
positioning said base plate under the patient's head; and
placing a transparent collapsible bag having an elastomeric band encircling an opening of said transparent bag over said arched ribs to position said elastomeric band adjacent said base plate and passing over the patient's chest to encapsulate the patient's head; and
attaching a remote vacuum source to an exhaust port affixed to said transparent bag located at a position elevated above the patient's head, said vacuum source evacuating air and aerosolized discharge from within said transparent bag.

15. The method of claim 14 wherein said base plate includes curved sections that prevent the transparent bag from sealing against said base plate, said elastomeric band extending around said mounting sockets and overlapping said curved sections of said base plate, said method further including the step of:
drawing air into said transparent bag from underneath said elastomeric band as air and aerosolized discharge are being evacuated from within said transparent bag.

16. The method of claim 15 wherein said elastomeric band includes sufficient stretch capabilities as to permit a medical attendant to lift said elastomeric band from above the patient's chest to permit access to the patient's head.

17. The method of claim 15 wherein said frame assembly when in said stored configuration has said curved ribs positioned against said base plate and pressed against mounting sockets on said base plate.

18. The method of claim 15 wherein said transparent bag is disposable when said protective hood is no longer being utilized, said frame assembly being formed of plastic can be cleaned appropriately and returned to said stored configuration awaiting a subsequent deployment.

19. The method of claim 18 wherein said curved ribs are tubular members having a circular diameter to facilitate cleaning.

20. The method of claim 19 wherein said frame assembly in said stored configuration is adapted for mounting on a vertical surface with a support bracket having a base member and orthogonal legs engagable with said base plate.
